(19) United States
(12) Patent Application Publication
(10) Pub. No.: US 2014/0039409 A1
(43) Pub. Date: Feb. 6, 2014

(54) Device for Injecting Adjustable Doses of Liquid Drug

(71) Applicants: Bo Radmer, Hilleroed (DK); Christian Peter Enggaard, Vejby (DK); Brian Mouridsen, Fredensborg (DK); Michael Frank Petersen, Solroed Strand (DK); Lars Ulrik Nielsen, Bagsvaerd (DK)

(72) Inventors: Bo Radmer, Hilleroed (DK); Christian Peter Enggaard, Vejby (DK); Brian Mouridsen, Fredensborg (DK); Michael Frank Petersen, Solroed Strand (DK); Lars Ulrik Nielsen, Bagsvaerd (DK)

(73) Assignee: Novo Nordisk A/S, Bagsvaerd (DK)

(21) Appl. No.: 13/976,849

(22) PCT Filed: Dec. 22, 2011
(86) PCT No.: PCT/EP11/73775

§ 371 Date: Oct. 25, 2013

Related U.S. Application Data
(60) Provisional application No. 61/429,610, filed on Jan. 4, 2011.

(30) Foreign Application Priority Data
Dec. 29, 2010 (EP) 10197269.3

(51) Int. Cl. A61M 5/315

(52) U.S. Cl. CPC A61M 5/3155
USPC 604/211

(57) ABSTRACT

The present invention relates to an injection device for administering doses of liquid drug. The injection device comprises a user operable dose adjustment structure configured to adjust, in a prepared state of the device, a dose of liquid drug of a first size to set a dose of a second size. The present injection device is particularly suitable for self-injection of liquid drugs such as insulin for treating diabetes by the user or patient.

The present invention relates to an injection device for administering doses of liquid drug. The injection device is particularly suitable for self-injection of liquid drugs, such as insulin for treating diabetes, by the user or patient.

### BACKGROUND OF THE INVENTION

Within some therapy areas the tendency of a patient to adhere to the prescribed therapy is dependent on the simplicity of the specific treatment regimen. For example, many people with type 2 diabetes are diagnosed with the disease at a relatively high age where they are less prone to accept a treatment that intervenes too much with their normal way of living. Most of these people do not like constantly being reminded of their disease and, as a consequence, they do not want to be entangled in complex treatment patterns or waste time on learning to operate cumbersome delivery systems. Basically, people with diabetes need to keep track of, and minimise, their glucose excursions. Insulin is a well-known glucose lowering agent which has to be administered parenterally to be effective in the body. At present, the most common way of administering insulin to a patient is by subcutaneous injections. Such injections have previously been performed using a vial and a syringe, but in recent years so-called injection devices, or injection pens, have gained more and more attention in the marketplace. Many people have found these injection devices easier to handle, particularly as they do not require the user to carry out a separate drug filling procedure before each injection.

In some prior art injection devices which are suitable for self-injection, the user has to set a desired dose size using a dose setting mechanism of the injection device and subsequently inject the previously set dose using an injection mechanism of the injection device. In this case the dose size is variable, i.e. the user must set a dose size which is suitable in the specific situation each time a dose is to be injected.

Other prior art injection devices are adapted to inject a dose of fixed size each time it is operated. In this case the user has to prepare the injection device in an appropriate manner to set the fixed dose size, using a dose setting or loading mechanism, and subsequently inject the dose using an injection mechanism.

WO 2009/092807 discloses an injection device which is simple and intuitive to handle and therefore easy for the patient to learn using. The disclosed injection device is loaded or prepared with a predetermined dose of liquid drug by the mounting of a protective cap with a twisting operation. The injection means is automatically disabled when the protective cap is mounted on the device and automatically enabled when the protective cap is dismounted from the device. The injection device automatically sets the predetermined dose when the protective cap is mounted so as to eliminate any risk of misadjusting the dose size by the user.

While the cap induced dose preparation of the disclosed injection device is desirable for its simplicity and minimization of the required number of manipulation steps for preparing the injection device, the fixed nature of the dose size may be impractical in certain situations. The user may need to adjust the size of the predetermined dose. The decision to adjust dose size is often taken right before the time of injection which may be many hours from the time where the injection device was prepared and the user condition such as a glucose level changed in the meantime. It would thus be desirable to provide an improved injection device which allows the user to make such a dose size adjustment of an already prepared or loaded injection device just before administration or injection. It would also be beneficial if the dose size adjustment can be made, in the prepared state, without spilling any drug

In the following disclosure of the present invention, aspects and embodiments will be described which address one or more of the above objects or which address objects apparent from the disclosure as well as from the description of exemplary embodiments.

### SUMMARY OF INVENTION

A first aspect of the invention relates to an injection device for administering doses of liquid drug, comprising a cartridge having a movable piston arranged therein and adapted to hold the liquid drug. The injection device further comprises a dose setting structure responsive to mounting of a removable cap to place the injection device in a prepared state with a dose of a first size and a user operable dose adjustment structure configured to, in the prepared state, adjust the dose of the first size to set a dose of a second size. An injection structure of the injection device comprises a piston rod coupled to the movable piston and configured to advance the piston a predetermined axial distance inside the cartridge from a first position in the prepared state to a second position in an unprepared state corresponding to delivery of the dose of the second size.

The present injection device allows the user to prepare the device with the dose of the first size by a simple mounting of the removable cap. The mounting may comprise a twisting or helical movement of the removable cap injection device therefore only requires a minimum of manipulation steps by the user. In accordance with the invention, the user operable dose adjustment structure is capable of adjusting the dose of the first size to set a dose of a second size in the prepared state of the injection device. This user is accordingly allowed to increase or decrease the size of a previously, i.e. at the time of mounting of the removable cap, set dose. This is beneficial because a decision to adjust the dose size is often taken right before the time of injection which may be many hours from the time where the injection device was prepared with the dose of the first size. The user condition such as a glucose level may have changed in the meantime. The user operable dose adjustment structure may be configured to adjust the dose size in discrete step(s) or continuously in a predetermined range within upper and lower limits for the second dose size. Depending on the user's or patient's condition at the time of drug administration, the user may choose to maintain an already set dose of the first size, in which case the first and second dose sizes are identical, or decrease/increase the dose of the first size to set a second dose of a different size.

During preparation or loading of the injection device, the movable piston is displaced to the first position in response to the mounting of the removable cap. A source of energy or mechanical force is preferably charged/loaded in that connection so that the device can be fired or unloaded and the injection made by stored energy delivered by the energy source.

The first position of the movable piston may be defined by a proximal clamping structure operatively coupled to the piston rod to retain the piston rod in the first position. In certain embodiments of the invention, the proximal clamping structure is fixedly attached to, or engraved in, the housing of the injection device. In certain embodiments the proximal clamping structure comprises a proximal shelf while other such embodiments comprise a circumferential slot, aperture or groove in the housing. The circumferential slot, aperture or groove may be configured to guiding a trajectory of a sliding element of the dose setting structure.

The second or distal position of the movable piston is preferably defined by a distal clamping structure operatively coupled to the piston rod to arrest the piston rod in the second distal position. The distal clamping structure defines an end-of-dose stop for the moveable piston. The moveable piston is preferably rigidly connected to the piston rod at least in an axial direction of the housing of the injection device so these are advanced the same predetermined axial distance during delivery of the dose of the second size. The dose setting structure is preferably configured to sequentially advancing the piston rod in axial direction for each new dose delivery.

In a number of useful embodiments of the injection device, the dose adjustment structure is configured to axially translate at least one of the distal clamping structure and the proximal clamping structure in the housing of the injection device to adjust the dose size. The dose adjustment structure is preferably configured to perform axial translation or movement of only a single one of the proximal and distal clamping structure to simply mechanical design. The axial translation of the proximal or distal clamping structure, in response to actuation of the dose adjustment structure, adjusts the predetermined axial distance by which the movable piston, and preferably the piston rod, is advanced during delivery of the dose of the second size. The user operable dose adjustment structure may comprise a circumferentially extending dose dial rotatably mounted about the housing of the injection device. The dose dial preferably comprises an inner threaded structure engaging the distal clamping structure or the proximal clamping structure to axially translate the distal or proximal clamping structure, respectively, by rotation of the dose dial. An outer surface of the dose dial may comprise a corrugated surface to improve the user's grip on the dial. In other embodiments, the dose dial is integrated with a proximally protruding injection button of the injection device as described in further detail in connection with the FIGS. 5a)-b).

A number of advantageous embodiments of the present injection device comprise a toothed sliding element adapted to engage mating teeth of a toothed axially extending section of the piston rod. The dose setting structure is configured to retain or arrest the toothed sliding element on the proximal clamping structure to set the first position of the piston rod. The mating teeth may be configured to allow unidirectional movement of toothed sliding element relative to the piston rod only. In this manner the toothed sliding element can move freely over the piston rod in proximal direction but is rigidly coupled to the piston rod in distal direction. Hence, the arrest of the sliding element on the proximal clamping structure also fixes a first or proximal position of the piston rod. In this embodiment, the dose adjustment structure may advantageously be configured to vary an axially extending geometry of the toothed sliding element to adjust the dose size. This may be accomplished by varying the axial position of a radially extending finger or protrusion of the sliding element that is configured for engagement with an axially fixed, i.e. non-translatable, distal clamping structure so as to adjust the predetermined axial distance by which the movable piston, and preferably the piston rod, is advanced during dose delivery.

According to a preferred embodiment of the invention, the user operable dose adjustment structure comprises a clutch mechanism configured to decouple the dose setting structure from the injection structure in the prepared state so as to allow increasing or decreasing the dose of the first size without spilling liquid drug. The clutch mechanism may comprise an axially biased and toothed nut, rotatably mounted on the piston rod. The teeth of the toothed nut are configured to selectively engage or disengage mating teeth of a toothed member of the dose setting structure. The toothed member of the dose setting structure is preferably formed as a separate intermediate element configured for engagement with the sliding element, but may alternatively be integrated with the toothed sliding element. In response to the user's mounting of the removable cap to prepare the injection device, the intermediate element may be axially displaced in proximal direction whereby the mating teeth of the intermediate element and toothed nut are disengaged, for example by the action of a compressed nut spring supplying the axial bias force to the toothed nut. By this step or action, the toothed sliding element, which forms part of the dose setting structure, is decoupled or disengaged from the toothed piston rod and toothed nut, which form part of the injection structure. According to this embodiment, the clutch mechanism may be configured to decouple the dose setting structure from the injection structure during an initial step or phase of the preparation or loading sequence of the injection device. This embodiment of the clutch mechanism prevents dosage spill caused by handling errors such as partial loading of the injection device by incomplete or partial mounting of the removable cap. In this situation, a subsequent dismounting of the removable cap would lead to unloading or firing of the injection device with accompanying drug spill if the piston rod had been in operative engagement with the toothed sliding element during the loading sequence. This type of undesired drug spill can be avoided by utilization of the above-mentioned configuration of the clutch mechanism.

In yet another embodiment of the invention, the clutch mechanism is formed by rotational engagement and disengagement of mating teeth structures formed in the toothed piston rod and the toothed sliding element. In this embodiment, the dose setting structure may be configured to rotate the toothed sliding element about the longitudinal axis of the housing during preparation of the device when the toothed sliding element reaches the first position. The toothed piston rod and the toothed sliding element may be engaged during axial translation of the toothed sliding element towards the first position during a preparation step of the injection device. The rotation of the toothed sliding element causes the disengagement between the mating teeth structures of the toothed piston rod and the toothed sliding element.

According to one such embodiment, the toothed piston rod comprises a first axially extending segment of teeth of a first radial height occupying a first predetermined circumferential surface of the toothed piston rod. A second axially extending segment of teeth of a second radial height, smaller than the first radial height, occupies a second predetermined circumferential surface of the toothed piston rod. The engagement and disengagement is preferably provided by rendering the radial height of the teeth of the second segment sufficiently small to avoid engagement with a radially protruding tooth or teeth of the sliding element when the sliding element is arrested in the first position after rotation. Consequently, the toothed sliding element is decoupled from the toothed piston rod and rendered in an axially translatable state. The first radial height of the teeth of the first axially extending segment is on the other hand set to a value which ensures engagement between the mating teeth structures of the toothed piston rod and toothed sliding element. Therefore, the toothed sliding element can be coupled to the toothed piston rod by a suitable rotatory movement thereof in connection with a firing step of the injection device. The angle of rotation of the sliding element may naturally be adapted to fit the respective angular extensions of the first and second predetermined circumferential surfaces to ensure appropriate engagement and disengagement between the mating teeth structures of the toothed piston rod and toothed sliding element is accomplished. In a number of preferred embodiments, the angle of rotation of the toothed sliding element relative to the toothed piston rod lies between 10 degrees and 180 degrees such as between 20 and 90 degrees.

The skilled person will understand that different types of energy sources may be applied for advancing the injection structure from the first to the second position in connection with dose delivery. In a preferred embodiment, the energy source driving the injection structure comprises a compression spring. The compression spring may be operatively coupled between the toothed sliding element and the housing of the injection device. The mounting of the removable cap to prepare the injection device causes axial compression of, and energy storage in, the compression spring due to the axial translation in proximal direction of the toothed sliding element.

In another preferred embodiment of the invention, the dose setting structure comprises a torsionally pre-tensioned spring operatively coupled between the sliding element and the housing. The torsionally pre-tensioned spring is configured to rotate the toothed sliding element into engagement with the proximal clamping structure at the first position of the toothed sliding element. In some of these embodiments, the proximal clamping structure may comprise the axially translatable shelf, the axial position of which can be moved by actuation of the dose adjustment structure to adjust the dose size. In other embodiments, the axial position of the proximal clamping structure may be fixed relative to the housing and comprise a circumferentially extending slot, groove or channel in an annular wall section of the housing. The circumferentially extending slot, groove or channel is configured to guide the rotation or rotary movement of the sliding element about the longitudinal housing axis for example by engagement with a matingly shaped finger or protrusion of the toothed sliding element. The rotation of the toothed sliding element ensures that the sliding element is safely retained or arrested at the proximal clamping structure in the prepared state so as to minimize any risk of unintended firing of the injection structure. The rotary movement ensures that this indication is provided only after the sliding element has been safely retained at the proximal clamping structure. In addition, the rotary movement of the toothed sliding element may be used to release, rotate and axially translate an injection button arranged in operative engagement with the toothed sliding element such as to indicate a prepared state of the injection device. The injection button may be axially translated a pre-set distance in proximal direction to move from a depressed state, indicating an unprepared state of the injection device, to a protruding state indicating the prepared state of the injection device. The dose setting means may be configured to render the injection button partly, or preferably entirely, contained within the housing contour in its depressed state. In the prepared state of the injection device, the injection button may in response to user actuation or depression thereof be configured to operatively engage and rotate the toothed sliding element a predetermined distance along the proximal clamping structure. The predetermined distance is preferably designed such that the rotation of the toothed sliding element is terminated when it reaches an axially extending slot in the annular wall section of the housing. The axially extending slot guides further distal advancement of the sliding element in axial direction driven by the spring force from the compressed helical spring.

In an advantageous embodiment, the above-mentioned torsionally pre-tensioned spring and the compression spring are integrally formed as a single helical compression spring, thus minimizing the number of separate components of the injection device and simplifying assembly or manufacturing processes. In this embodiment, the axially extending slot in the annular wall section of the housing may be adapted to guide the axial movement in proximal direction of the toothed sliding element in connection with preparation of the injection device. The rotational movement of the toothed sliding element may, as mentioned above, be guided by the circumferentially extending slot in the annular wall structure. The axially extending slot and the circumferentially extending slot may be combined to form an L-shaped slot structure.

### BRIEF DESCRIPTION OF THE DRAWINGS

Preferred embodiments of the invention will be described in additional detail in connection with the appended drawings, in which:

FIGS. 1 a) and 1 b) are respective central axial cross-sectional views of an injection device in accordance with a first embodiment of the invention,

FIGS. 2 a) and 2 b) illustrate first and second steps, respectively, of a preparation and firing sequence of the injection device depicted on FIG. 1,

FIGS. 2 c) and d) illustrate third and fourth steps, respectively, of the preparation and firing sequence of the injection device depicted on FIG. 1,

FIGS. 2 e) and f) illustrate fifth and sixth steps, respectively, of the preparation and firing sequence of the injection device depicted on FIG. 1,

FIGS. 3 a) and 3 b) depict a user operable dose adjustment structure in respective cross-sectional views to illustrate steps of a dose adjustment function of the injection device depicted on FIG. 1,

FIGS. 4 a) and 4 b) are respective central cross-sectional views of a user operable dose adjustment structure of an injection device in accordance with a second embodiment of the invention,

FIGS. 5 a) and 5 b) are respective central axial cross-sectional views of an injection device in accordance with a third embodiment of the invention,

FIGS. 6 a) and 6 b) illustrate first and second steps, respectively, of a preparation and firing sequence of the injection device depicted on FIG. 5 in accordance with the third embodiment of the invention,

FIGS. 6 c) and 6 d) illustrate third and fourth steps, respectively, of the preparation and firing sequence of the injection device depicted on FIG. 5 in accordance with the third embodiment of the invention,

FIGS. 6 e) and 6 f) illustrate fifth and sixth steps, respectively, of the preparation and firing sequence of the injection device depicted on FIG. 5 in accordance with the third embodiment of the invention,

FIG. 6 g) illustrates a seventh step of the preparation and firing sequence of the injection device depicted on FIG. 5 in accordance with the third embodiment of the invention,

FIG. 7 a) is a central cross-sectional view of a user operable dose adjustment structure of the injection device depicted on FIG. 5 in accordance with the third embodiment of the invention,

FIG. 7 b) is a perspective view of a sliding element with a variable axial dimension mounted in the user operable dose adjustment structure depicted on FIG. 5 a),

FIG. 7 c) is a central cross-sectional view of an end-of-content feature of the injection device depicted on FIG. 5 a)-b) under normal operating conditions; and

FIG. 7 d) is a central cross-sectional view of the end-of-content feature of the injection device depicted on FIG. 5 a)-b) in an end of content mode.

### DETAILED DESCRIPTION OF PREFERRED EMBODIMENTS

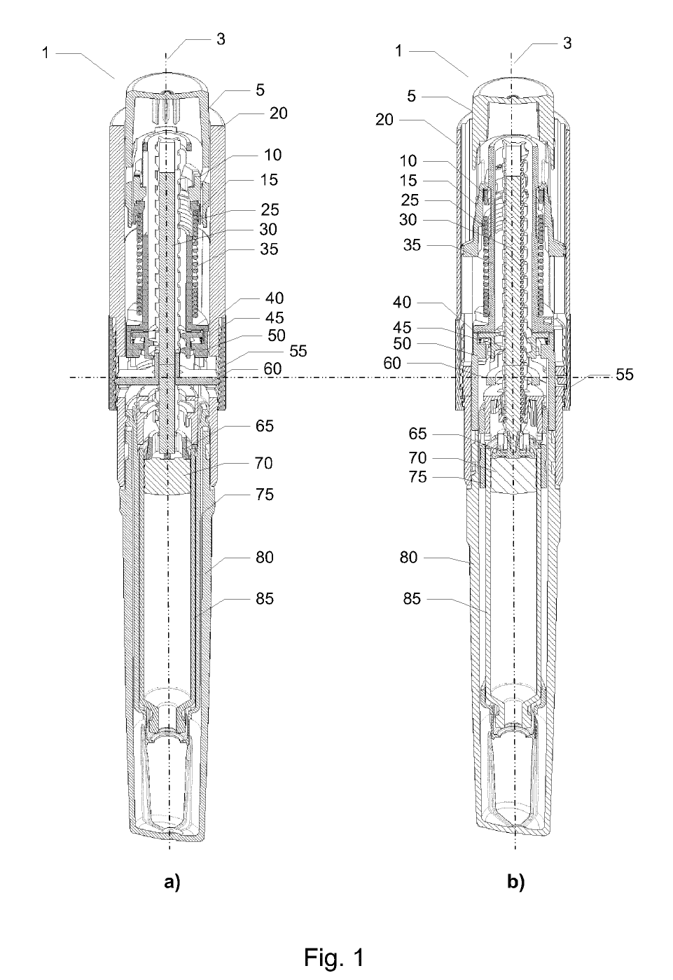

FIGS. 1 a) and 1 b) are respective central axial cross-sectional views of an injection device 1 in accordance with a first embodiment of the invention wherein the depicted cross-sectional views are made at an angular separation of 90 degrees by rotation of the injection device 1 about a central longitudinal axis 3.

The injection device 1 is illustrated in a prepared or loaded state ready to deliver a dose of liquid drug to a user or patient by self-administration. The injection device 1 comprises a tubular housing 20, a cartridge 85 holding a volume of liquid drug and an injection button 5 protruding axially from the tubular housing 5. An injection needle (not shown) is attached to a distal portion of the cartridge 85 for subcutaneous injection of a predetermined dose of liquid drug in accordance with the user's setting of a dose size. A toothed elongate piston rod 30 is rigidly attached or coupled to a movable piston 70 via a piston foot 65. The movable piston 70 is arranged within an interior volume of the cartridge 85. Consequently, advancing the toothed piston rod 30 a predetermined distance axially in distal direction will cause a corresponding axial displacement of the movable piston 70 and cause a dose of the liquid drug to be expelled via the injection needle. A dose setting structure is responsive to mounting of a removable cap 80 to place the injection device 1 in the prepared or loaded state with a dose of liquid drug of a first size. The dose setting structure comprises an intermediate element in the form of a pusher 50 configured for engagement with the removable cap 80 and axially displaceable by mounting of the removable cap 80. The pusher 50 is configured for engaging a sliding element 35 and axially displacing the sliding element 35 in proximal direction, i.e. towards the injection button 5. The displacement of the sliding element 35 leads to a loading or preparation of the injection device 1 with a dose of liquid drug of the first size as explained in further detail below. The sliding element 35 comprises teeth arranged on an inner surface and configured to engaging mating teeth of the toothed elongate piston rod 30. The mating teeth of the sliding element 35 and toothed elongate piston rod 30 are configured to solely allow unidirectional displacement of the sliding element 35 relative to the toothed elongate piston rod 30 or piston rod. Only proximal displacement of the sliding element 35 relative to the piston rod 30 is allowed. Consequently, the mating teeth of piston rod 30 and the sliding element 35 are brought into operative engagement when the latter moves in an opposite direction, i.e. a distal direction towards the second or distal position defined by an adjustable shelf 60.

The sliding element 35 is coupled to a helical compression spring 25 co-axially arranged around a tubular neck or portion of the sliding element 35. The compression spring 25 is torsionally pre-tensioned and compressible by proximal displacement of the sliding element 35 during a loading sequence or operation of the injection device 1. The loading sequence therefore causes potential energy or spring force to be stored in the compression spring 25 for release in connection with forward firing or advancement of the piston rod 30 and movable piston 70 during injection of the set dose of liquid drug. One end portion of the compression spring 25 engages the sliding element 35 and an opposing end portion engages a spring base 15 rigidly attached to the housing 20.

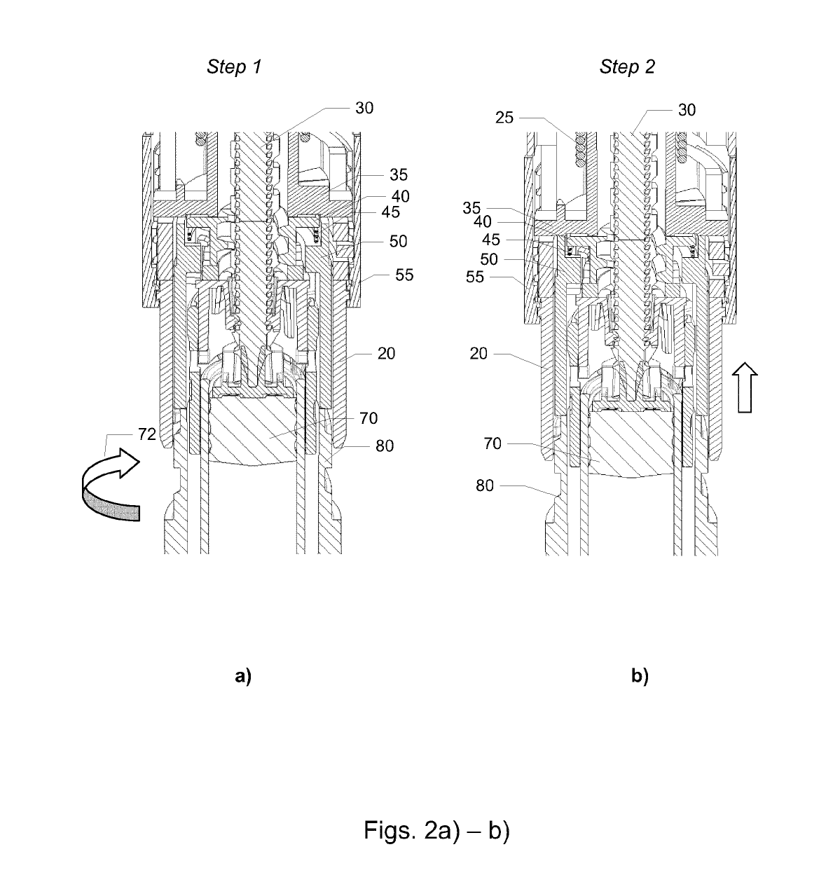

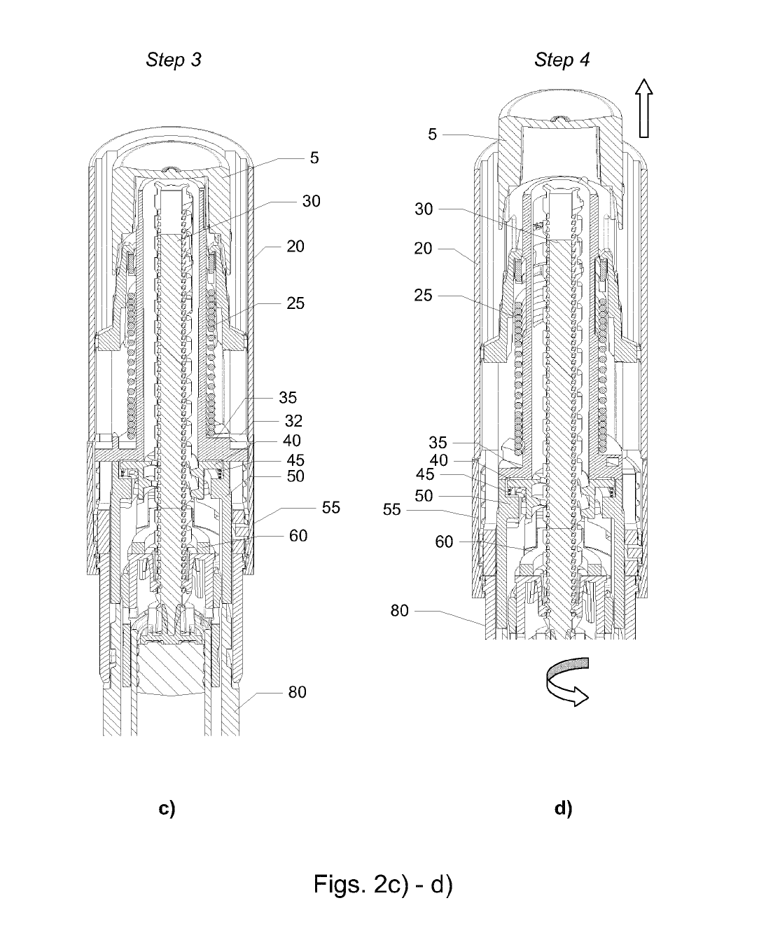

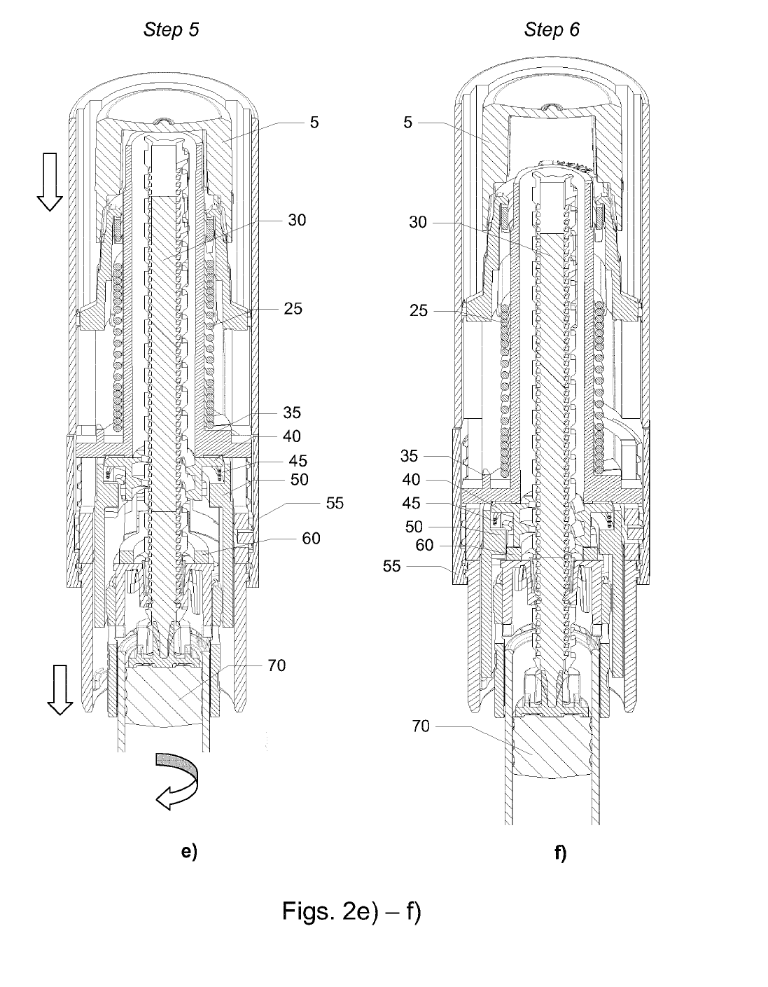

The injection device 1 furthermore comprises user operable dose adjustment structure comprising dose dial 55 and configured to, in the prepared or load state, increase or decrease the dose of the first size to set a dose of a second size. The dose dial 55 is configured to adjust a position, relative to the housing 20, of an axially translatable distal clamping structure in form of the adjustable shelf 60 so as to vary the dose size in accordance with the user's adjustment of the dose dial 55 as explained in further detail below. The user operable dose adjustment structure additionally comprises a clutch mechanism configured to decouple the dose setting structure from the injection structure in the prepared state. The clutch mechanism comprises the pusher 50 which is configured to selectively engage or disengage a toothed nut 40 operatively coupled to the piston rod 30 as as explained in further detail below in connection with FIGS. 2 b)-2 f).

The injection button 5 is configured to noticeably project from the housing 1 in the prepared state of the injection device 1 as depicted on FIGS. 1 a) and 1 b) to indicate a current state of the injection device to the user or patient. By depression of the injection button 5 a firing sequence is initiated where the sliding element 35 is released from a a proximal clamping structure and the piston rod 30 advanced from a first or proximal position relative to the housing 1 to the second or distal position in an unprepared or unloaded state of the injection device 1. An axial distance between the first and second positions accordingly corresponds to the delivery of the dose of the second size.

The clutch mechanism comprises a toothed nut 40, a nut spring 45 and a toothed inner peripheral surface of the pusher 50. The clutch mechanism is configured to decouple the dose setting structure from the injection structure in the prepared state of the injection device so as to allow dose adjustment in the prepared state by actuation of the dose dial 55 without advancing the toothed piston rod 30 and the moveable piston 70 and spillage of liquid drug as explained in further detail below.

FIG. 2 a) illustrates a first step of the loading and firing sequence where the injection device 1 depicted on FIG. 1 is loaded or prepared. In connection with the first step, loading or preparation is initiated by the user by twisting the replaceable cap 80 onto the injection device 1 following a helical trajectory as indicated by arrow 72.

FIG. 2 b) illustrates the second step of the loading and firing sequence where the injection device 1 is loaded or prepared. The pusher 50 is the first portion of the dose setting structure to move in response to the mounting of the removable cap 80. As previously explained, the piston rod 30 can only move in one direction, distally, relative to the housing 20 of the injection device. This effect is created by a pair of one way snaps mounted in the housing 20 and engaging the teeth on piston rod 30. The pusher 50 is rotationally locked to the housing 20. The pusher 50 is axially displaced by the twisting operation of the removable cap 80, but the toothed nut 40, which is rotatably mounted on the piston rod 30, stands still due to an inner thread (not shown) that engages a mating thread on the piston rod 30. The disengagement between the toothed nut 40 and the pusher 50 allows the toothed nut 40 to rotate as it is pushed proximally/upwards by the pusher 50 and the nut spring 45. The toothed nut 40 will start to rotate due to the threaded non-locking interface with the mating teeth on the piston rod 30.

The teeth of the toothed nut 40 are arranged around a circumferential outer perimeter of the toothed nut 40. The teeth of the pusher 50, which are arranged on an inner tubular surface of the pusher 50 as explained above, are forced to disengage the mating teeth on the toothed nut 40 by this translation due to an axially directed biasing force supplied by the nut spring 45. The toothed nut 40 can now rotate freely about the piston rod 30. In effect, the dose setting structure has been decoupled from the injection structure because the piston rod 30 is no longer operatively coupled to the sliding element 35.

FIG. 2 c) illustrates the third step of the loading and firing sequence where the injection device 1 is undergoing loading or preparation. In this step, the helical compression spring 25 is compressed and loaded with axial spring force and a torque. The axial force is later on used to supply dose delivery force or energy during a user initiated firing or dose delivery sequence as explained below. The torque is obtained by torsionally pre-tensioning the helical compression spring 25 and using this torque to radially rotate the sliding element 35 into engagement with a proximal clamping structure at a first or proximal position of the piston rod 30. The helical twisting of the removable cap 80 is configured to axially translate the pusher 50 and the sliding element 35 to a first position guided by an axial slot (not shown) in a tubular wall section of the housing 20. At the first position, a circumferentially and essentially horizontally extending slot or channel 32 in the tubular wall section guides rotary movement of the sliding element 35 about a longitudinal housing axis 3 (refer to FIG. 1a)). The combination of the axial slot and the circumferentially extending slot 32 forms an L-shaped slot in the housing 20. The toothed nut 40 is free to rotate in the non self-.locking thread engaging the piston rod 30 when the pusher 50 and the sliding element 35 translate.

FIG. 2 d) illustrates the fourth step of the loading and firing sequence where the injection device 1 is loaded or prepared. The sliding element 35 will rotate because of the freedom in the housing 20 and the torque generated by the pretensioned helical compression spring 25. Furthermore, the injection button 5 is rotated and axially translated, in response to the axial displacement and rotation of the sliding element 35, from an unloaded or unprepared state indicated by its non-protruding placement inside the housing 20 of injection device to a loaded or prepared state indicated by the protruding placement depicted in FIG. 2 d). Consequently, after completion of step 4, the injection device is rendered in the prepared state with the removable cap 80 mounted on the injection device. The sliding element 35 rests in the circumferentially extending slot 32 in the housing 20 with the sliding element 35 decoupled from the pusher 50. It is now possible to adjust the axial position of the adjustable shelf 60 which effectively defines the second position or end-step of the sliding element 35. Since the first position of the sliding element, as defined by the circumferentially extending slot 32, remains fixed, the predetermined axial distance which the sliding element 35 and piston rod 30 travels during the dose delivery is varied. This leads in turn to the desired adjustment of the size of the initially set dose.

FIG. 2 e) illustrates the fifth step of the loading and firing sequence where the injection device 1 is fired or unloaded. The sliding element 35 rests in the circumferentially extending slot 32 in the housing 20 when the removable cap 80 is removed by the user as explained above. Furthermore, the pusher 50 is configured to translate a small distance axially and engage with the toothed nut 40 so as to rotationally lock to, or engage, the toothed nut 40 by virtue of the mating sets of teeth arranged on the pusher 50 and the toothed nut 40 as explained above. The engagement can be made in a manner where the interacting teeth make an incremental rotation of the toothed nut 40 to compensate for possible tolerances caused by small variations in the user's mounting process of the replaceable cap 80. This will improve the dose accuracy.

When the injection button 5 is depressed as indicated by the arrow adjacent to the button 5, the sliding element 35 will also be forced to rotate due to a helical spiralling movement of the injection button 5 under engagement with an end surface of the sliding element 35. The rotary movement of the sliding element 35 is guided by the circumferentially extending slot 32 and continues until the sliding element 35 reaches the axial slot in the housing 20.

FIG. 2 f) illustrates the sixth step of the loading and firing sequence where the injection device 1 is fired or unloaded. When the sliding element 35 reaches the axial slot in the housing 20, the sliding element 35 is translated axially in distal direction because of the axial force generated by the compressed helical compression spring 25. The toothed nut 40 will translate axially in a corresponding manner because of the locked engagement with the pusher 50. The toothed nut 40, which is coupled to the piston rod 30 by the threaded interface, will subsequently advance the piston rod 30 and the movable piston 70 inside the cartridge 85 to make a dosing in accordance with the user selected dose size. The depicted end-of-dose or second position of the piston 70, and corresponding end position of the piston rod 30, is defined or set by the adjustable shelf 60 operating as the distal clamping structure or end-stop. The adjustable shelf 60 interrupts any further distal advancement of the piston 70 once the adjustable shelf 60 engages the toothed nut 40.

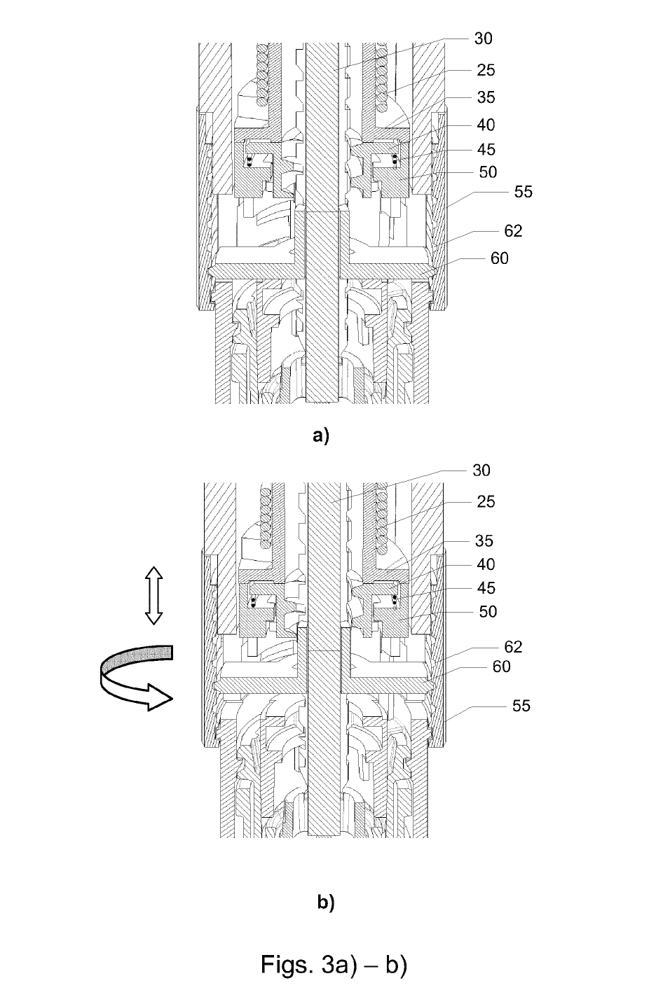

FIGS. 3 a) and 3 b) depict the user operable dose adjustment structure in respective cross-sectional views and illustrate functionality of the dose adjustment structure of the injection device 1 depicted on FIG. 1. FIG. 3 a) illustrates a current state of the injection device 1 after completion of step 4 above, i.e. the current state is the prepared state where the dose already has been set to a first size. This first size will correspond to a previously injected dose size. The user is now able to adjust the dose of the first size to set a dose of a second size in accordance with his/hers current condition by axially moving the adjustable shelf 60 either distally or proximally. The adjustable shelf 60 is translatable inside the housing 20. The dose dial 55 is configured for rotation about the housing 20 but is unable to move axially or translate relative to the housing 20. The dose dial 55 comprises an internal thread 62 which mates to a corresponding circumferential end structure of the adjustable shelf 60 as illustrated. The adjustable shelf 60 is accordingly forced to move axially in response to rotation of the dose dial 55. Since the sliding element 35 is rests safely on the proximal clamping structure (the circumferentially extending slot 32), the axial position of the adjustable shelf 60 can be safely adjusted without inducing any corresponding displacement of the piston rod 30 and movable piston 70. Therefore, without causing any spillage of the liquid drug. On the other hand, the adjustment of the axial position of the adjustable shelf 60 leads to the desired dose size adjustment because any positional change alters the axial distance of travel of the piston rod 30 and piston movable piston 70 during the firing sequence or dose delivery.

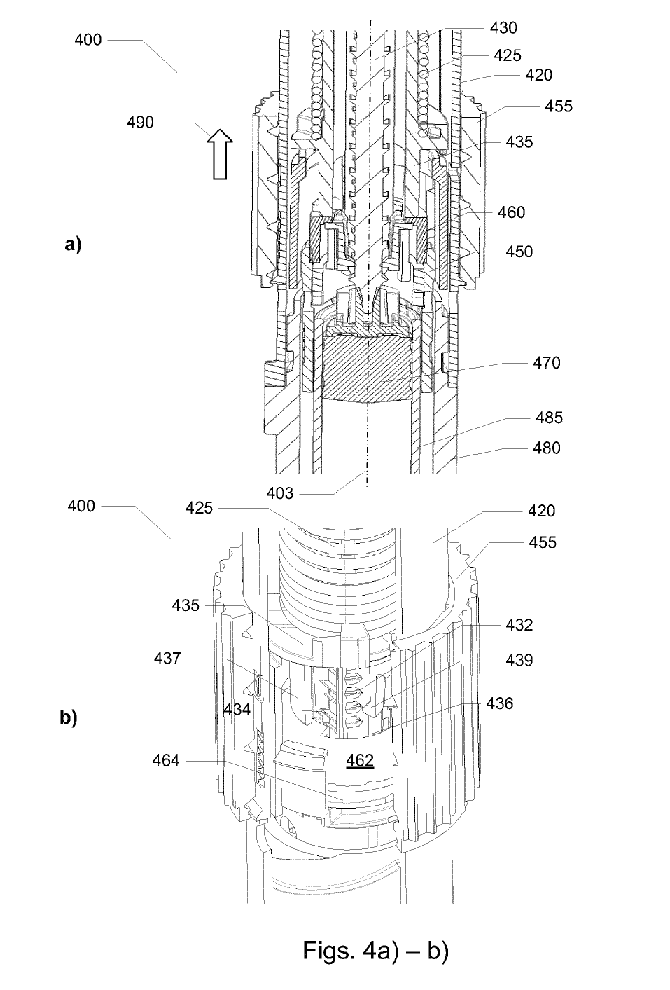

FIG. 4 a) is a central cross-sectional view of a user operable dose adjustment structure of an injection device 400 in accordance with a second embodiment of the invention. The injection device 400 has many features in common with the above-described first embodiment of the injection device. However, the dose adjustment structure of the first embodiment utilized axial movement of a distal clamping structure (the adjustable shelf 60) to adjust the desired dose size in the prepared state of the injection device 1. The present injection device 400 utilizes axial movement of a proximal clamping structure (for example an adjustable shelf) to adjust the dose size in the prepared state of the injection device 400. A distal clamping structure or end-stop remains fixed. Furthermore, the present injection device 400 utilizes a different type of clutch mechanism to decouple a dose setting structure from an injection structure in the prepared state of the injection device where the clutch mechanism is formed integrally with a toothed piston rod 430 and a sliding element 435.

FIG. 4a) shows the injection device 400 in the prepared state ready to deliver a dose of liquid drug to a user or patient by self-administration when the user depresses an injection button (not shown) similar in structure to the one depicted on FIG. 1. The injection device 400 comprises a tubular housing 420, a cartridge 485 holding a volume of liquid drug. The injection button (not shown) is protruding axially from a proximal end of the housing 420. An injection needle (not shown) is attached to a distal portion of the cartridge 485 for subcutaneous injection of a predetermined dose of liquid drug in accordance with the user's setting of a dose size. The toothed elongate piston rod 430 is rigidly attached to a movable piston 470. The movable piston 470 is arranged within an interior volume of the cartridge 485. Consequently, advancing the piston rod 430 a predetermined distance axially in distal direction will cause a corresponding axial displacement of the piston 470 and cause the liquid drug to be expelled via the injection needle (not shown). A dose setting structure is responsive to mounting of a removable cap 480 to place the injection device 400 in the illustrated prepared or loaded state with a dose of liquid drug of a first size. The dose setting structure comprises a pusher 450 configured for engagement with the removable cap 480 and axially displaceable by mounting of the removable cap 480. The pusher 450 is configured for engaging a sliding element 435 and axially displaces the sliding element 435 in proximal direction, i.e. the direction indicated by arrow 490 which is towards the injection button. The displacement of the sliding element 435 in proximal direction leads to the loading of the injection device 400 with a dose of liquid drug of a first size as explained in further detail below. The sliding element 435 comprises teeth engaging mating teeth of the piston rod 430. The mating teeth of the sliding element and piston rod are configured to solely allow unidirectional displacement in the proximal direction of the sliding element 435 relative to the piston rod 430. Consequently, the piston rod 430 is advanced together with the sliding element 435 when the latter is advanced in the opposite direction, i.e. a distal direction towards a second or distal position defined by the fixed distal clamping structure or end-stop as explained below in connection with FIG. 4 b). The sliding element 435 is coupled to, or engages, a helical compression spring 425 co-axially arranged around a tubular portion or neck of the sliding element 435. An opposite end of the helical compression spring 425 is operatively coupled to the housing 420 in similar manner to the first embodiment of the injection device. The injection device 400 furthermore comprises a user operable dose adjustment structure actuated by the dose dial 455 and configured to, in the prepared or load state, increase or decrease the dose of the first size to set a dose of a second size. The dose dial 455 is configured to adjust a position of an axially translatable proximal clamping structure in form of a proximal adjustable shelf 460 so as to vary the set dose size in accordance with the user's adjustment of the dose dial 455 as explained in further detail below in connection with FIG. 4 b).

FIG. 4 b) is a perspective view of the user operable dose adjustment structure of the injection device 400 in partial cross-section. As previously mentioned, the injection device 400 is placed in its prepared state where the sliding element 435 rests on the proximal adjustable shelf 460 and the helical compression spring 425 is axially compressed. The sliding element 435 comprises an axially extending finger 437 which rests on an upper plane surface 462 of the adjustable proximal shelf 460 so as to define a first or proximal position of the piston rod 430. The piston rod 430 comprises a first axially extending segment of teeth 434 extending across a first predetermined circumferential surface of the toothed piston rod 430. The teeth have a first radial height. Another axially extending segment of teeth 432 is placed adjacently to the first axially extending segment of teeth 434 so as to occupy a second predetermined circumferential surface of the piston rod 430. The teeth of the second segment 432 have a radial height which is smaller than the first radial height. A radially protruding tooth 439 of the sliding element 430 is configured for engagement with individual teeth of the first axially extending segment of teeth 434. However, in the illustrated state the radially protruding tooth 439 is placed at the teeth of the second segment 432 which have a radial height sufficiently small to avoid engagement with the radially protruding tooth 439 of the sliding element 435. Consequently, the sliding element 435 is decoupled from the piston rod 430 and rendered axially translatable by movement or adjustment of the axial position of the upper plane surface 462 of the adjustable proximal shelf 460. The axial position of the sliding element 435 can accordingly be adjusted without adjusting the axial position of the piston rod 430 and the moveable piston 470 so as to avoid drug spillage during dose size adjustment.

During firing of the injection device 400, the sliding element 435 is firstly rotated about the longitudinal housing axis 403 of the injection device which causes the axially extending finger 437 or finger to travel across the upper plane surface 462 of the adjustable proximal shelf 460 in rotary movement until the finger 437 reaches a slot or aperture 436 in the upper plane surface 462. During this rotary movement of the sliding element 435, the radially protruding tooth 439 is rotated as well until it is placed at the first axially extending segment of teeth 434 or first segment of teeth of the toothed piston rod 430. Due to the larger radial height of the teeth of the first segment of teeth 434, the radially protruding tooth 439 of the sliding element is now brought into a locked engagement with the teeth of the first segment of teeth 434. Consequently, the sliding element 435 is now coupled to the piston rod 430 such that piston rod will translate axially together with the sliding element 435 in the distal direction towards the second position of the piston rod and piston. The skilled person will appreciate that an integrally formed clutch mechanism resides in the described cooperation between the sliding element 435 and the piston rod 430. This integrally formed clutch mechanism operates by rotational engagement and disengagement of the mating teeth structures 432, 434, 439 formed in respective ones of the toothed piston rod 430 and the slider element 435.

Once the finger 437 has reached the slot or aperture 436 in the adjustable upper shelf 460, the spring force or energy stored in the axially compressed helical compression spring 425 will advance the sliding element 435 and the piston rod 430 (now brought into engagement by the clutch mechanism) in axial direction. The sliding element 435 and the piston rod 430 will advance together until the finger 437 contacts or engages a non-adjustable or fixed lower shelf 464 which blocks further axial advancement of the sliding element 435 and the piston rod 430. The fixed lower shelf 464 therefore defines an end-of-dose or the second position of the piston 470 and corresponding second or distal position of the piston rod 430 after delivery of the set dose size.

The depicted user operable dose adjustment structure of the injection device 400 allows the user to increase or decrease a dose of a first size to set a dose of a second size by axially moving the adjustable proximal shelf 460 either distally or proximally to respectively decrease or increase the dose size. The adjustable proximal shelf 460 is translatable inside the housing 420. The tubular dose dial 455 is configured for rotation about the housing 420 but unable to move axially relative to the housing 420. The dose dial 455 comprises an internal thread which mates to a corresponding circumferential end structure of the adjustable shelf 460 in a manner similar to the above-described dose dial 55 (refer to FIG. 3 b)) of the first embodiment. The adjustable shelf 460 is accordingly forced to move axially in response to rotation of the dose dial 455. Even though the sliding element 435 rests on the upper plane surface 462 of the adjustable shelf 460 as illustrated on FIG. 4b), the sliding element 435 is decoupled from the piston rod 430 by the operation of the clutch mechanism as described above. Therefore, the position of the adjustable shelf 460 can be adjusted axially without inducing any corresponding movement of the piston rod 430 and movable piston 470. The position of the adjustable shelf 460, and therefore the dose size, can accordingly be adjusted without spillage of liquid drug. Furthermore, the adjustment of the axial position of the adjustable shelf 460 leads to the desired dose size adjustment because the positional change alters the axial distance of travel of the piston rod 430 and piston movable piston 470.

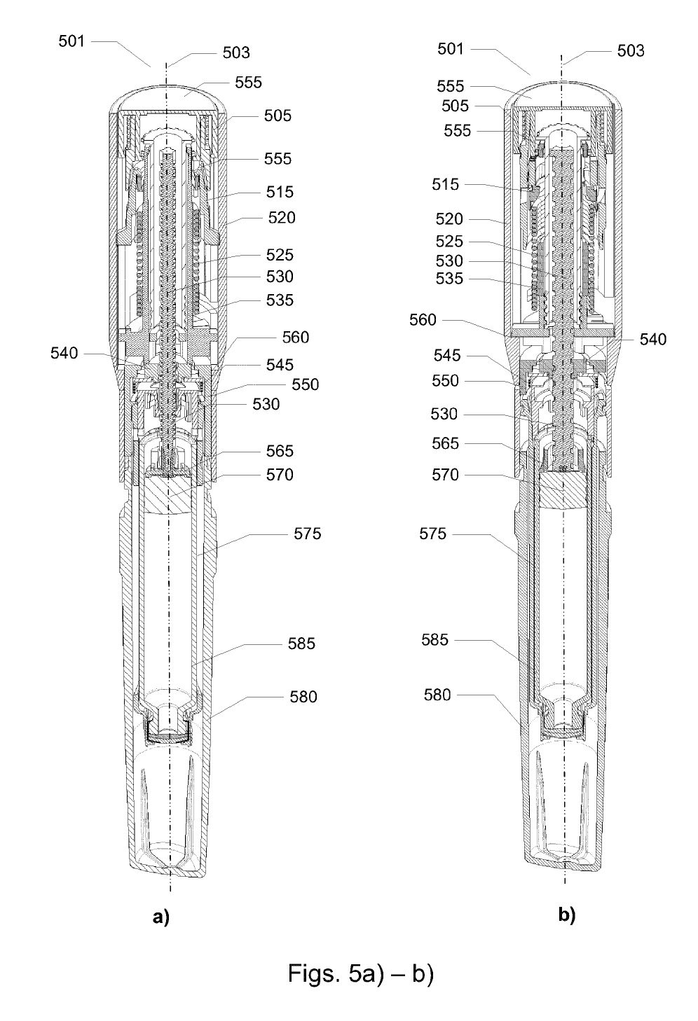

The loading or preparation of the injection device 400 is generally similar to the one for the first injection device 1 described above in connection with FIGS. 2a)-d) albeit with a different operation of the clutch mechanism. The helical compression spring 425 is torsionally pre-tensioned and compressible by proximal displacement of the sliding element 435 during the loading sequence of the injection device 400. The torque obtained from the torsionally pre-tensioned helical compression spring 425 is used to rotate the the sliding element 435 once the adjustable proximal shelf 460 is reached and bring the finger 437 into engagement with the upper plane surface 462 in connection with the mounting of removable cap 480 by helical twisting. Once the removable cap 480 has been mounted, the injection device 400 is automatically rendered in a prepared state with a dose of the first size where the finger 437 of the sliding element 435 rests safely on the upper plane surface 462 of the adjustable upper shelf 460. FIGS. 5 a) and 5 b) are respective central axial cross-sectional views of an injection device 501 in accordance with a third embodiment of the invention wherein the depicted cross-sectional views are shown at an angular separation of 90 degrees by rotation of the injection device 501 about a central longitudinal axis 503.

The injection device 501 has many features in common with the above-described first embodiment of the injection device 1 on FIG. 1. The dose adjustment structure of the first embodiment utilized axial movement of a distal clamping structure (the adjustable shelf 60) to adjust the desired dose size in the prepared state of the injection device 1. In contrast, the dose adjustment structure of the present injection device 501 is configured to vary an axially extending geometry of a toothed sliding element 535 to adjust the dose size in the prepared state. In the present injection device 501, the respective axial positions of a distal clamping structure and a proximal clamping structure remain fixed. Furthermore, a user operable dose dial is integrated with an injection button of the injection device 501 as explained in further details below.

The injection device 501 is illustrated in an unprepared or unloaded state after delivery of a dose of liquid drug to a user or patient by self-administration. The injection device 501 comprises a tubular housing 520, a cartridge 585 holding a volume of liquid drug and an injection button 505 protruding axially from the housing 520. An injection needle (not shown) is attached to a distal portion of the cartridge 585 for subcutaneous injection of a predetermined dose of liquid drug in accordance with the user's setting of a dose size. A toothed elongate piston rod 530 is rigidly attached to a movable piston 570 via a piston foot 565. The movable piston 570 is arranged within an interior volume of the cartridge 585. Consequently, advancing the toothed piston rod 530 a predetermined distance axially in distal direction will cause a corresponding axial displacement of the piston 570 and cause a dose of the liquid drug to be expelled via the injection needle. A dose setting structure is responsive to the mounting of a removable cap 580 to place the injection device 501 in a prepared or loaded state with a dose of liquid drug of a first size. The dose setting structure comprises a pusher 550 configured for engagement with the removable cap 580 and axially displaceable by mounting of the removable cap 580. The pusher 550 is configured for engaging a sliding element 535 and axially displaces the sliding element 535 in proximal direction, i.e. towards the injection button 505. The displacement of the sliding element 535 leads to a loading or preparation of the injection device 501 with a dose of liquid drug of the first size as explained in further detail below. The sliding element 535 comprises teeth engaging mating teeth of the toothed elongate piston rod 530 or piston rod. The mating teeth of the sliding element 35 and the piston rod 530 are configured to solely allow unidirectional displacement in proximal direction of the sliding element 535 relative to the toothed elongate piston rod 530 or piston rod. Consequently, the piston rod 530 is advanced together with the sliding element 535 when the latter moves in an opposite direction, i.e. a distal direction towards the second or distal position defined by the fixed distal clamping structure formed as a cut-out or shelf in the housing 520.

The sliding element 535 is coupled to a helical compression spring 525 co-axially arranged around a tubular portion of the sliding element 535. The compression spring 525 is torsionally pre-tensioned and compressible by proximal displacement of the sliding element 535 during a loading sequence of the injection device 501. The loading sequences therefore causes potential energy or compression force to be stored in the helical compression spring 525 for release in connection with forward firing or advancement of the piston rod 530 and movable piston 570 during injection of the liquid drug. One end portion of the compression spring 525 engages the sliding element 535 and an opposing end portion engages a spring base 515 rigidly attached to the housing 520.

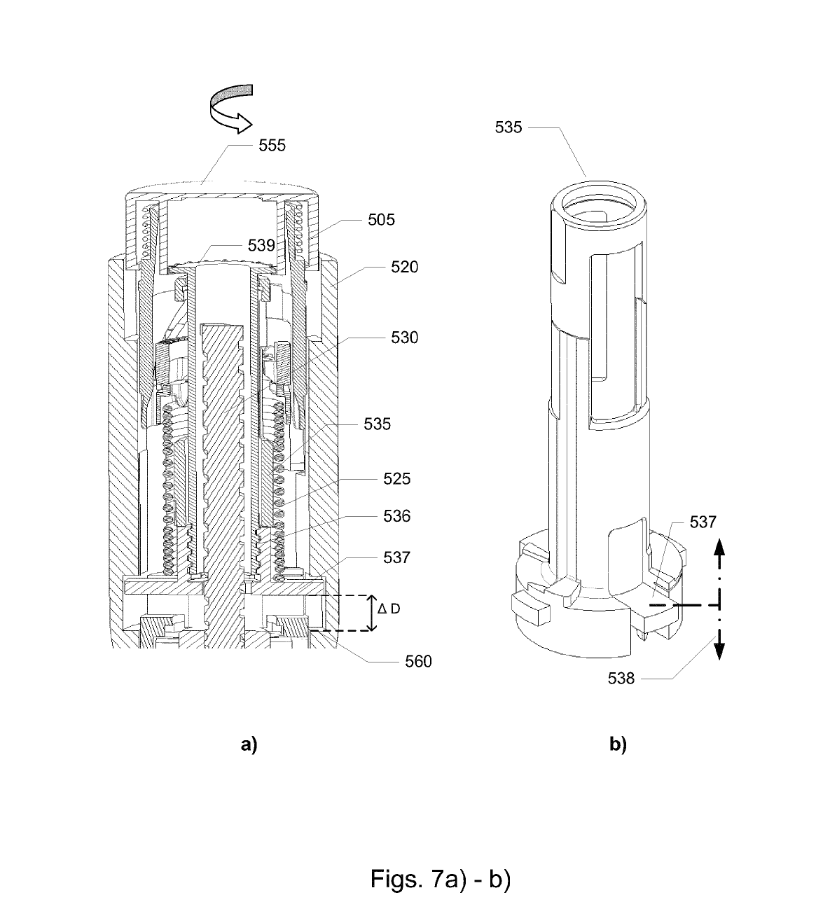

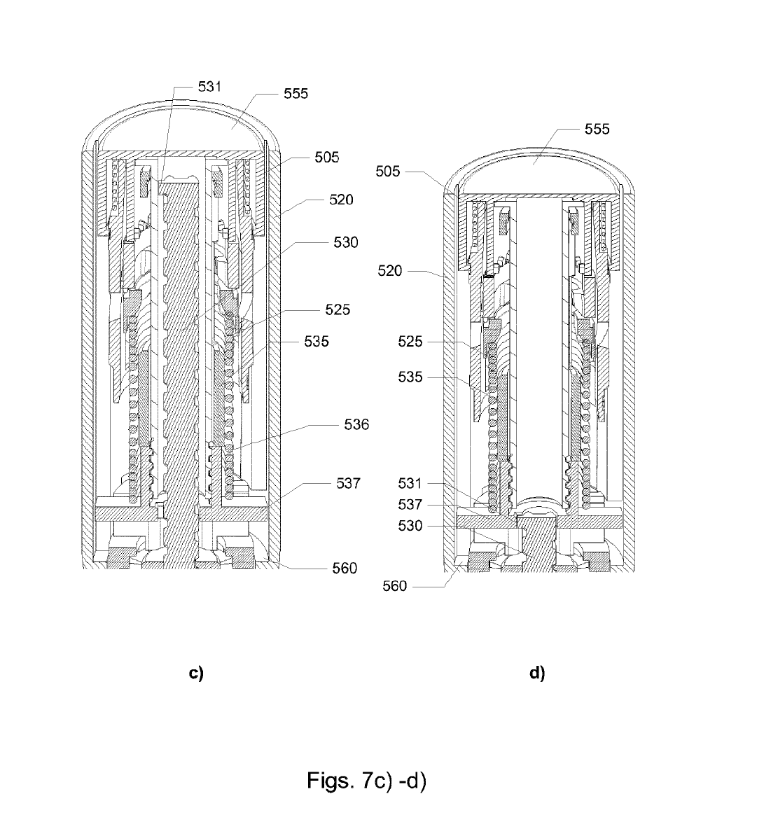

The injection device 501 furthermore comprises user operable dose adjustment structure or dose dial 555 configured to, in the prepared or load state, increase or decrease the dose of the first size to set a dose of a second size. The dose dial 555 is configured to adjust an axial position of an axially translatable finger (refer to item 537 on FIGS. 7a)-b) so as to vary the dose size in accordance with the user's adjustment of the dose dial 555 as explained in further detail below. A fixed position distal clamping structure 560 or distal shelf is formed in the housing 520 and defines an end-stop for advancement of the axially translatable finger of the sliding element 535.

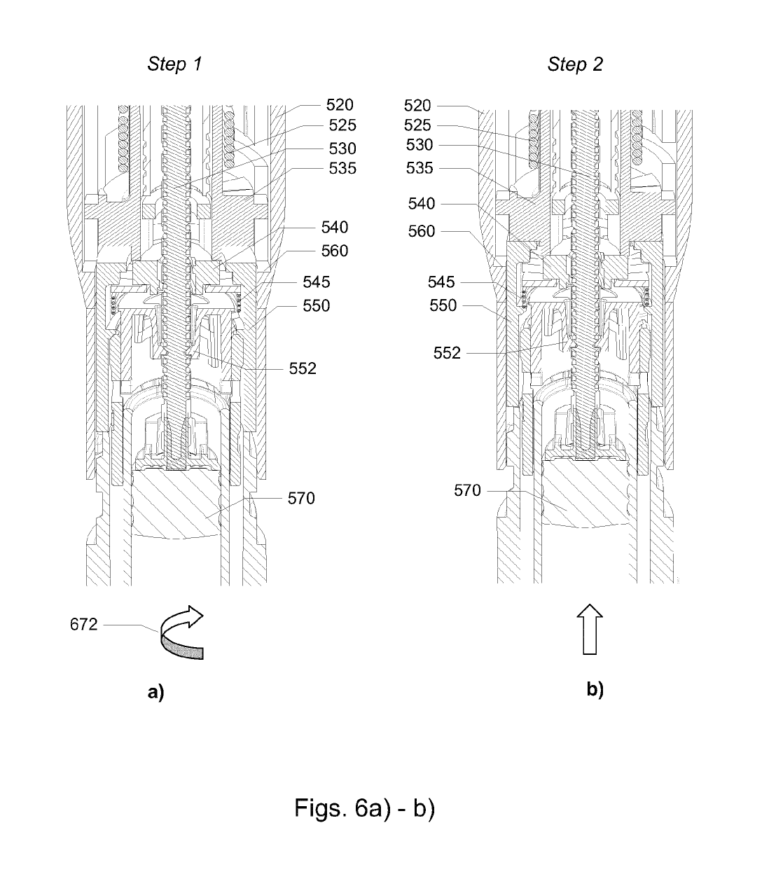

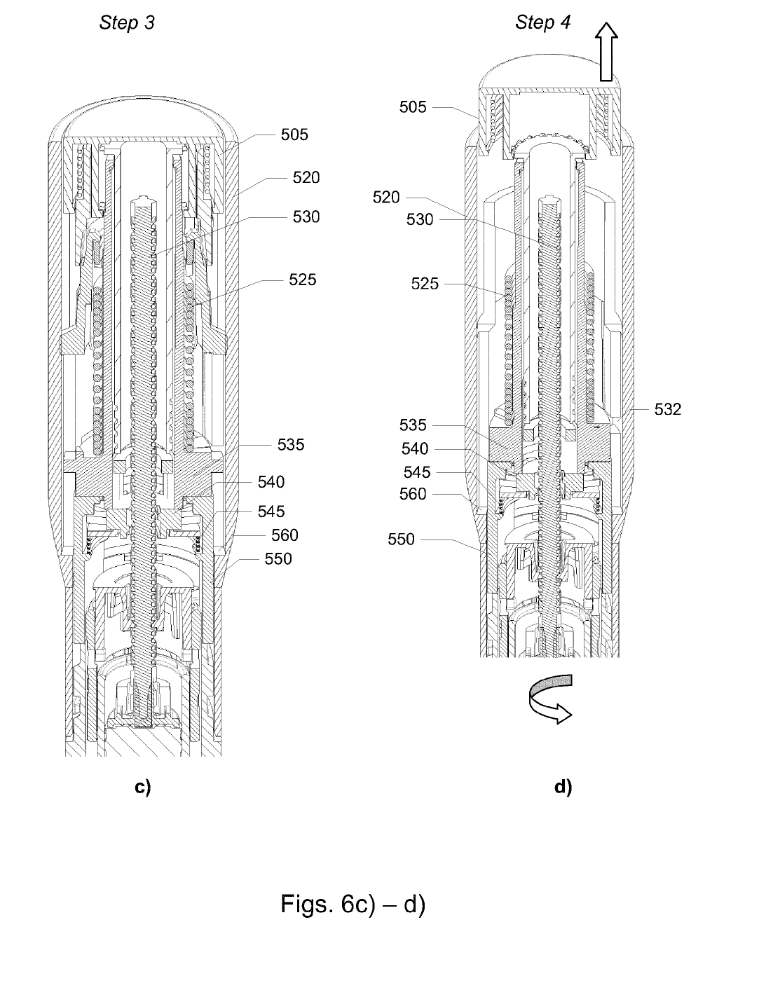

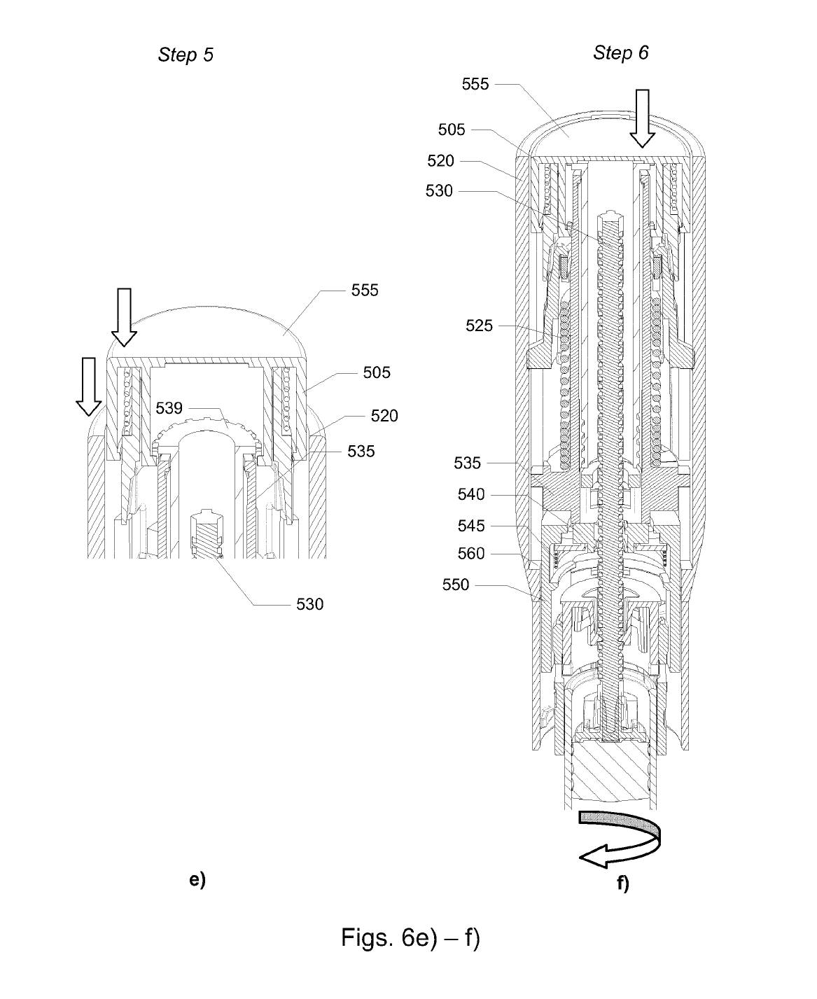

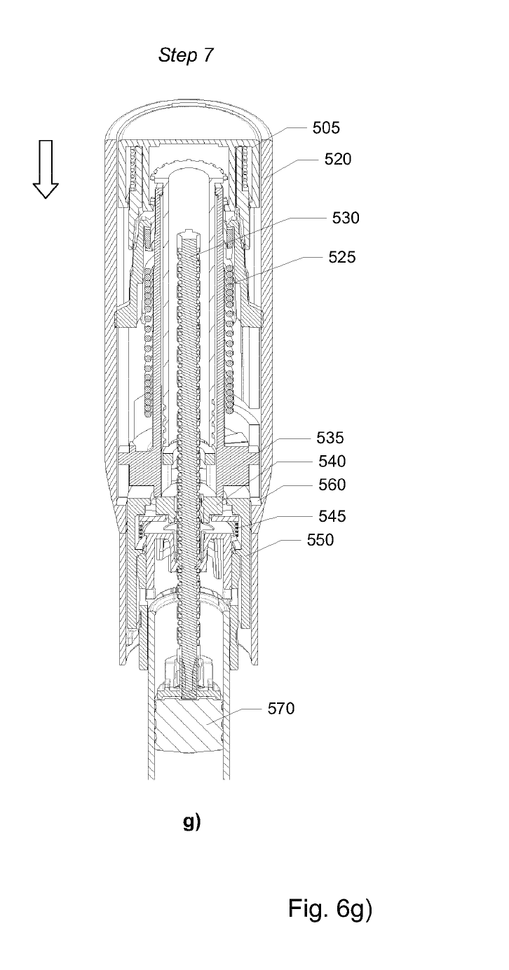

The user operable dose adjustment structure additionally comprises a clutch mechanism configured to decouple the dose setting structure from the injection structure in the prepared state. The clutch mechanism comprises a pusher 550 configured to selectively engage or disengage a toothed nut 540 operatively coupled to the piston rod 530 as as explained in further detail below in connection with FIGS. 6 b)-6 f).

The injection button 505 is configured to noticeably project from the housing 520 in the prepared state of the injection device 501 as depicted on FIG. 6 d) to indicate a current state of the injection device 501 to the user or patient. By depression of the injection button 505 in the prepared state, a firing sequence is initiated where the sliding element 535 is released from the proximal clamping structure and the piston rod 530 advanced from a first or proximal position relative to the housing 501 to the second or distal position in an unprepared or unloaded state of the injection device 501. A predetermined axial distance between the first and second positions accordingly corresponds to the delivery of the dose of the second size.

The clutch mechanism comprises a toothed nut 540, a nut spring 545 and a toothed inner peripheral surface of the pusher 550. The clutch mechanism is configured to decouple the dose setting structure from the injection structure in the prepared state of the injection device so as to allow dose adjustment in the prepared state by actuation of the dose dial 555 without advancing the toothed piston rod 530 and piston 570 and spillage of liquid drug as explained in further detail below.

FIG. 6 a) illustrates a first step of a loading and firing sequence of the injection device 501 where the device is loaded or prepared. In connection with the first step, loading or preparation is initiated by the user by twisting the replaceable cap 580 onto the injection device 501 following a helical trajectory as indicated by arrow 672.

FIG. 6 b) illustrates a second step of a loading and firing sequence of the injection device 501 where the device is loaded or prepared. The pusher 550 is the first portion of the dose setting structure to move in response to mounting of the removable cap 580. As previously explained, the piston rod 530 can only move axially in one direction, a distal direction, relative to the housing 520 of the injection device. This effect is created by a pair of one way snaps 552 mounted in the housing 520 and engaging the teeth on piston rod 530. The pusher 550 is rotationally locked to the housing 520. The pusher 550 is axially displaced by the twisting operation of the removable cap 580, but the toothed nut 540, which is rotatably mounted on the piston rod 530, stands still due to an inner thread (not shown) that engages a mating thread on the piston rod 530. The disengagement between the toothed nut 540 and the pusher 550 allows the toothed nut 540 to rotate as it is pushed proximally/upwards by the pusher 550 and the nut spring 545. The toothed nut 540 will start to rotate about the piston rod 530 due to the threaded non-locking interface with the mating teeth on the piston rod 530.

The teeth of the toothed nut 540 are arranged around a circumferential outer perimeter of the toothed nut 540. The teeth of the pusher 550, which are arranged on an inner tubular surface of the pusher 550 as explained above, are forced to disengage the mating teeth on the toothed nut 540 by this translation due to an axially directed biasing force supplied by the nut spring 45. The toothed nut 540 can now rotate freely about the piston rod 530. In effect, the dose setting structure has been decoupled from the injection structure because the piston rod 530 is no longer operatively coupled to the sliding element 535.

FIG. 6 c) illustrates third step of the loading and firing sequence of the injection device 501 where the device is loaded or prepared. In this step, the helical compression spring 525 is compressed and loaded with axial force and a torque. The axial force is later on used to supply dose delivery force or energy during a user initiated firing or dose delivery sequence as explained below. The torque is obtained by torsionally pre-tensioning the helical compression spring 525 and using this torque to rotate the sliding element 535 about the axis of the housing 501 into engagement with a proximal clamping structure at a first or proximal position of the piston rod 530. The helical twisting of the removable cap 580 is configured to axially translate the pusher 550 and the sliding element 535 to a first position guided by an axial slot 532 (depicted on FIG. 6 d)) in a annular wall section of the housing 520. At the first position, the circumferentially extending slot or channel 532 in the tubular wall section guides rotary movement of the sliding element 535 about the longitudinal housing axis 503. The combination of the axial slot and the circumferentially extending slot 532 forms an L-shaped slot in the housing 520. The toothed nut 540 is free to rotate in the non-self locking thread engaging the piston rod 530 when the pusher 550 and the sliding element 535 translate.

FIG. 6 d) illustrates the fourth step of the loading and firing sequence where the injection device 501 is loaded or prepared. The sliding element 535 will rotate because of the freedom in the housing and the torque generated by the helical compression spring 525. Furthermore, the injection button 505 is rotated and axially translated, in response to the axial displacement and rotation of the sliding element 535, from an unloaded or unprepared state indicated by its non-protruding placement inside the housing 520 of injection device to a loaded or prepared state indicated by the protruding placement depicted in FIG. 6 d). Consequently, after completion of step 4, the injection device 501 is rendered in its prepared or loaded state with the removable cap mounted on the injection device 501. The sliding element 535 rests in the circumferentially extending slot 532 in the housing 520 with the sliding element 535 decoupled from the pusher 550. It is now possible to adjust an axial position of the axially translatable finger (refer to item 537 on FIGS. 7a)-b) movably mounted in the sliding element 535. The adjustment of the axial position of the finger leads to an adjustment of the size of the dose as explained in further detail below. The adjustment of the dose size is accomplished by actuating the dose dial 555 by the user or patient as explained in further detail below in connection with FIGS. 7a)-b).

FIG. 6 e) illustrates the fifth step of the loading and firing sequence of the injection device 501 where the device 501 fired or unloaded. The sliding element 535 rests in the circumferentially extending slot 532 in the housing 520 when the removable cap 580 is removed by the user as explained above. When the injection button 505 is depressed as indicated by the arrow adjacent to the button 505, a first movement of the injection button 505 will disengage mating teeth structures arranged on the injection button 505 and dose adjustment structure. The injection button 505 comprises a radially and inwardly projecting toothed annular structure coupled to mating teeth extending radially outwardly from a tubular proximal end section 539 of the sliding element 535. After the first movement, the sliding element 535 is able to rotate freely.

FIG. 6 f) illustrates the sixth step of the loading and firing sequence where the injection device 501 is fired or unloaded. In connection with the first movement of the injection button 505, the pusher 550 will translate a small distance axially and engage with the toothed nut 450 so as to rotationally lock to, or engage, the toothed nut 540 by virtue of the mating sets of teeth arranged on the pusher 550 and the toothed nut 540 as explained above.

When the injection button 505 is depressed further as indicated by the arrow adjacent to the button 505, the sliding element 535 is also forced to rotate due to a helical spiralling movement of the injection button 505 under engagement with the end surface of the sliding element 535. The rotary movement of the sliding element 535 is guided by the circumferentially extending slot 532 and continues until the sliding element 535 reaches the axial slot (not shown) in the housing 520.

FIG. 6 g) illustrates the sixth step of the loading and firing sequence where the injection device 501 is fired or unloaded. When the sliding element 535 reaches the axial slot in the housing 520, the sliding element 535 is translated axially in distal direction because of the axial force generated by the compressed helical compression spring 525. The toothed nut 540 will translate axially in a corresponding manner because of the locked engagement with the pusher 550. The toothed nut 540 will subsequently advance the piston rod 530 axially and distally since these components are axially locked to each other. The advancement of the piston rod 530 will lead to a corresponding advancement of the movable piston 570 inside the cartridge 585 so as to make a dosing in accordance with the user selected dose size. The depicted end-of-dose or second position of the piston rod 530 is reached once the axially translatable finger (not shown) reaches the distal shelf or end-stop 560 engraved into the housing 520 as explained below in further detail in connection with FIG. 7a).

FIG. 7 a) is a central cross-sectional view of a user operable dose adjustment structure of the injection device 501 depicted on FIG. 5. As previously explained, the dose dial 555 is integrated with the injection button 505. Adjustment of an already set first dose size, i.e. set during the above-described loading steps of the injection device, is achieved by rotation of the dose dial 555. As previously explained, a radially and inwardly projecting toothed annular structure of the injection button is coupled to the radially and outwardly projecting teeth arranged on the tubular proximal end section 539 of the sliding element 535. Rotation of the dose dial 555 causes axial translation or movement of the position of the finger 537 as indicated by the axially pointing arrows 538 on FIG. 7 b) due to a threaded interface 536 between a lower tubular portion of the sliding element 535 and the finger 537 in connection with the allocated space in the housing for axial movement of the latter as illustrated. Axial movement of the finger 537 changes the axial distance AD between the finger 537 and the fixed position distal shelf 560, which defines an end-stop for the finger 537 at engagement as explained above. The end-stop also functions as an end-stop for the residual part of the sliding element 535 and therefore defines a second or distal position of the piston rod 530 after dose delivery due to the interlocked engagement between the sliding element 535 and piston rod 530 during distal advancement in connection with the above-described firing or delivery sequence. In the illustrated situation, the axial distance travelled by the piston rod 530 from the first or proximal position in the prepared state to the second position in the unprepared state is

AD and corresponds to the delivery of the set dose after a possible user adjustment of an initially set first dose size by manipulation of the dose dial 555. Therefore, adjustment of the axial position of the finger 537 will adjust the travel distance AD of the piston rod 530 in a corresponding manner and adjust the size of the delivered dose of liquid drug. FIG. 7 c) is a central cross-sectional view of an end-of-content feature of the injection device depicted on FIGS. 5 a)-b) under normal operating conditions. Under the normal operating conditions, the piston rod 530 is sequentially advanced in axial direction for each new dose delivery. As explained above, the sliding 535 element rotates about the central axis when it reaches the circumferentially extending slot or channel in the tubular wall section of the housing in connection with the loading sequence and the firing sequence. However, in the end of content mode depicted on FIG. 7 d), the sliding element 535 is prevented from further rotation. A projection 531 arranged in an end portion of the piston rod 530 engages a mating cut out in the finger 537 of the sliding element 535 and locks the piston rod 530 for rotation. If the removable cap is mounted on the injection device by the user in this end of content mode, the sliding element 535 will translate and seek to rotate when it is possible. However, the finger 537 forms part of the sliding element 535 and is rotationally locked thereto. If the injection device is in the end of content mode, the sliding element 535 is blocked for rotation because the finger 537 and the piston rod 530 are unable to rotate. Since the injection button 505 is advanced to its projecting position, indicating a prepared or loaded state of the injection device, by rotation of the sliding element 535, the injection button 505 will stay in the illustrated depressed state (not protruding from the housing 520) and indicate to the user that the injection device has been emptied.

While the above-described injection devices have been designed as disposable devices, the skilled person will understand that the each of the disclosed injection devices by suitable modifications could be provided with suitable means for cartridge replacement to provide a durable injection device.

1. An injection device for administering doses of liquid drug, comprising:
a cartridge having a movable piston arranged therein and adapted to hold the liquid drug,
a dose setting structure responsive to mounting of a removable cap to place the injection device in a prepared state with a dose of a first size,
a user operable dose adjustment structure configured to, in the prepared state, adjust the dose of the first size to set a dose of a second size,
an injection structure comprising a piston rod coupled to the movable piston and configured to advance the piston a predetermined axial distance inside the cartridge from a first position in the prepared state to a second position in an unprepared state corresponding to delivery of the dose of the second size.

2. An injection device according to claim 1, wherein the proximal position of the movable piston is defined by a proximal clamping structure operatively coupled to the piston rod to retain the piston rod in a first position.

3. An injection device according to claim 1, wherein the second position of the movable piston is defined by a distal clamping structure operatively coupled to the piston rod to arrest the piston rod in a distal position.

4. An injection device according to claim 2, wherein the dose adjustment structure is configured to axially translate at least one of the distal clamping structure and the proximal clamping structure in a housing of the injection device to adjust the dose size.

5. An injection device according to claim 2, further comprising of a toothed sliding element adapted to engage mating teeth of a toothed axially extending section of the piston rod; and
the dose setting structure being configured to arrest the toothed sliding element on the proximal clamping structure to set the first position of the piston rod.

6. An injection device according to claim 5, wherein the dose adjustment structure is configured to vary an axially extending geometry of the toothed sliding element to adjust the dose size.

7. An injection device according to claim 5, wherein the user operable dose adjustment structure comprises a circumferentially extending dose dial rotatably mounted about the housing of the injection device,
wherein the dose dial comprises an inner thread structure engaging the distal clamping structure or the proximal clamping structure to axially translate the distal or proximal clamping structure, respectively, by rotation of the dose dial.

8. An injection device according to claim 1, wherein the user operable dose adjustment structure comprises a clutch mechanism configured to decouple the dose setting structure from the injection structure in the prepared state so as to allow adjustment of the dose of the first size without spilling liquid drug.

9. An injection device according to claim 1, wherein the clutch mechanism is configured to decouple the dose setting structure from the injection structure during a loading sequence of the injection device.

10. An injection device according to claim 9, wherein the clutch mechanism comprises an axially biased and toothed nut rotatably mounted on the piston rod; and
wherein teeth of the toothed nut are configured to selectively engage or disengage mating teeth of a toothed member of the dose setting structure.

11. An injection device according to claim 8, wherein the clutch mechanism is formed by rotational engagement and disengagement of mating teeth structures formed in the toothed piston rod and in the slider element.

12. An injection device according to claim 11, wherein the toothed piston rod comprises:
a first axially extending segment of teeth of a first radial height occupying a first predetermined circumferential surface of the toothed piston rod,
a second axially extending segment of teeth of a second radial height smaller than the first radial height and occupying a second predetermined circumferential surface of the toothed piston rod.

13. An injection device according to claim 5, wherein the injection structure further comprises a compression spring operatively coupled between the toothed sliding element and the housing; and
wherein mounting of the removable cap to load the injection device causes axial compression of, and energy storage in, the compression spring.

14. An injection device according to claim 5, wherein the dose setting structure comprises a torsionally pre-tensioned spring operatively coupled bet ween the sliding element and the housing and configured to rotate the toothed sliding element into engagement with the proximal clamping structure at the first position of the toothed sliding element.

15. An injection device according to claims 13, wherein the torsionally pre-tensioned spring and the compression spring are integrally formed as a single helical compression spring.
